(19) United States
(12) Patent Application Publication
(10) Pub. No.: US 2017/0302882 A1
(43) Pub. Date: Oct. 19, 2017

(54) SYSTEM AND METHOD FOR INTERFACING IMAGE MANAGER TO MONITOR FOR MEDICAL IMAGING APPLICATIONS

(71) Applicant: Ampronix, Inc., Irvine, CA (US)

(72) Inventor: Nausser Fathollahi, Irvine, CA (US)

(21) Appl. No.: 15/488,375

(22) Filed: Apr. 14, 2017

Related U.S. Application Data
(60) Provisional application No. 62/324,190, filed on Apr. 18, 2016.

(51) Int. Cl. H04N 7/08; A61B 5/00; H04N 7/18

(52) U.S. Cl. CPC H04N 7/08; H04N 7/181; A61B 5/7425; A61B 5/7445

(57) ABSTRACT

The disclosure relates to monitor interface for interfacing an image manager to a monitor. The interface includes a set of input ports for receiving a set of input image signals from the image manager, respectively. The interface includes a set of image multiplexers configured to multiplex pairs of the set of input image signals to generate output image signals, respectively. The interface further includes a set of output ports configured to produce the output image signals, respectively. The set of output ports are configured to be compliantly mated with a set of input ports of a monitor. The monitor is configured to simultaneously render images on separate portions of a screen based on the output image signals, respectively. From the image manager perspective, the monitor interface appears as it is a legacy monitor, and provides authentication information pertaining to the legacy monitor to the image manager.

### CROSS-REFERENCE TO RELATED APPLICATION

This application claims the benefit of the filing date of Provisional Application Ser. No. 62/324,190, filed on Apr. 18, 2016, which is incorporated herein by reference.

### FIELD

Aspects of the present disclosure relate generally to medical imaging systems, and more particularly, to a system, apparatus, and method for interfacing an image manager to a monitor.

### BACKGROUND

Medical imaging systems typically include a set of medical imaging devices, an image manager for routing image signals from the set of medical imaging devices to a monitor based on a user input, and the monitor for simultaneously rendering multiple images on a screen based on the image signals, respectively. In such systems, the image manager is configured to interface with a predetermined monitor or a set of predetermined monitors.

However, monitors for medical imaging applications are continuously being designed for providing improved performance and/or additional features. When a medical provider desires to upgrade to such new monitors, the provider often has to purchase a new image manager configured to operate with the new monitor. However, such image manager is typically expensive. Thus, it is desirable for medical providers to upgrade to new medical imaging monitors without the need to upgrade the corresponding image managers.

### SUMMARY

The following presents a simplified summary of one or more embodiments in order to provide a basic understanding of such embodiments. This summary is not an extensive overview of all contemplated embodiments, and is intended to neither identify key or critical elements of all embodiments nor delineate the scope of any or all embodiments. Its sole purpose is to present some concepts of one or more embodiments in a simplified form as a prelude to the more detailed description that is presented later.

An aspect of the disclosure relates to an interface for interfacing an image manager to a monitor. The interface comprises a housing configured to be securely attached to a monitor; a set of input ports configured to receive a set of input image signals from the image manager, wherein the set of input ports are disposed on a first surface of the housing; at least one image multiplexer configured to multiplex at least a pair of the set of input image signals to generate at least one output image signal, wherein the at least one image multiplexer is situated within the housing; and at least one output port disposed on a second surface of the housing, wherein the at least one output port is configured to compliantly mate with at least one input port of the monitor while the housing is securely attached to the monitor, wherein the at least one output port is configured to produce the at least one output image signal, respectively.

Another aspect of the disclosure relates to a medical imaging system comprising a set of medical imaging devices configured to generate first and second pairs of input image signals concerning a subject undergoing a medical treatment or diagnosis; an image manager configured to route the first and second pairs of input image signals from a first set of inputs ports to a first set of output ports based on a user input; and a monitor interface.

The monitor interface includes a housing; a second set of input ports coupled to the first set of output ports of the image manager, respectively, wherein the second set of input ports are disposed on a first surface of the housing; a pair of image multiplexers disposed within the housing, wherein the pair of image multiplexers is configured to multiplex the first and second pairs of input image signals to generate first and second output image signals, respectively; and first and second output ports disposed on a second surface of the housing, wherein the first and second output ports are configured to produce the first and second output image signals, respectively.

The medical imaging system further includes a monitor including first and second input ports compliantly mated with the first and second output ports of the monitor interface, respectively. The monitor is configured to simultaneously render first and second images on first and second portions of a screen based on the first and second output image signals, respectively.

Another aspect of the disclosure relates to a medical imaging apparatus comprising a monitor interface and a monitor. The monitor interface comprises a housing; a first set of input ports configured to receive a set of input image signals, respectively, wherein the first set of input ports are disposed on a first surface of the housing; a set of image multiplexers configured to multiplex pairs of the set of input image signals to generate a set of output image signals, respectively, wherein the set of image multiplexers are enclosed within the housing; and a set of output ports configured to produce the set of output image signals, respectively, wherein the set of output ports are disposed on a second surface of the housing.

The monitor includes a second set of input ports compliantly mated with the set of output ports of the interface, respectively. The monitor is configured to simultaneously render a set of images on separate portions of a screen based on the set of output image signals, respectively.

To the accomplishment of the foregoing and related ends, the one or more embodiments include the features hereinafter fully described and particularly pointed out in the claims. The following description and the annexed drawings set forth in detail certain illustrative aspects of the one or more embodiments. These aspects are indicative, however, of but a few of the various ways in which the principles of various embodiments may be employed and the description embodiments are intended to include all such aspects and their equivalents.

### BRIEF DESCRIPTION OF THE DRAWINGS

FIG. 1 illustrates a block diagram of an exemplary medical imaging system in accordance with an aspect of the disclosure.

FIG. 2 illustrates a block diagram of another exemplary medical imaging system in accordance with another aspect of the disclosure.

FIG. 3 illustrates a block diagram of an exemplary monitor interface in accordance with another aspect of the disclosure.

FIG. 4 illustrates a block diagram of another exemplary medical imaging system in accordance with another aspect of the disclosure.

FIG. 5 illustrates a block diagram of another exemplary monitor interface in accordance with another aspect of the disclosure.

FIG. 6 illustrates a rear perspective view of another exemplary monitor interface in accordance with another aspect of the disclosure.

FIGS. 7A-7C illustrate front perspective, rear perspective, and rear disassembled views of another monitor interface in accordance with another aspect of the disclosure.

FIGS. 8A-8C illustrate front perspective, rear perspective, and rear disassembled views of another monitor interface in accordance with another aspect of the disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts.

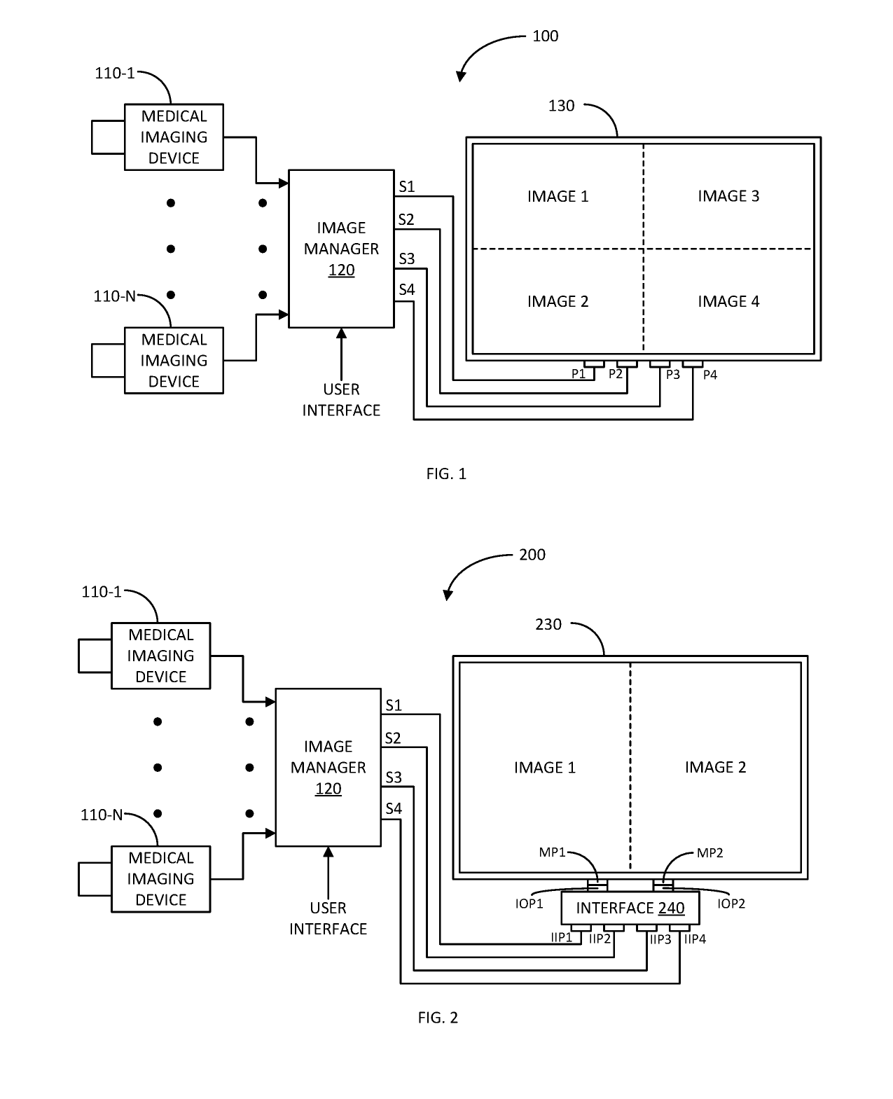

FIG. 1 illustrates a block diagram of an exemplary medical imaging system 100 in accordance with an aspect of the disclosure. The system 100 includes a set of one or more medical imaging devices 110-1 to 110-N, an image manager 120, and a monitor 130. The set of one or more medical imaging devices 110-1 to 110-N are configured to generate a set of one or more image signals of a subject (e.g., patient) undergoing a medical procedure (e.g., angiography, heart surgery, etc.) or diagnosis, respectively. The one or more image signals may include light images, X-ray images, computed tomography (CT) images, mammography images, molecular resonance imaging (MRI), ultrasound images, and others. The images may be still-pictures, video, or any combination thereof.

The set of one or more image signals generated by the set of one or more medical imaging devices 110-1 to 110-N are applied to an image manager 120. The image manager 120 includes a user interface to allow a user (e.g., a medical personnel, physician, surgeon, nurse, etc.) to direct any of the set of one or more image signals from the set of one or more medical imaging devices to a set of one or more output image signals S1, S2, S3, and S4. As an example, the image manager 120, via the user interface, may be configured to: direct the image signal from medical imaging device 110-1 to output image signal S2; direct the image signal from medical imaging device 110-2 to output image S1; direct the image signal from medical imaging device 110-5 to output image signal S4; and direct the image signal from medical imaging device 110-N to output image signal S3. Accordingly, the image manager 120 allows the user to select the region (e.g., quadrant) of the monitor screen on which each corresponding image is to be displayed.

The output image signals S1-S4 of the image manager 120 are applied to corresponding inputs P1-P4 of the monitor 130, respectively. For example, the image signals S1-S4 may be sent to the monitor 130 by way of appropriate cabling or other transmission medium. The monitor 130 renders “Image 1” associated with image signal S1 applied to input port P1 in the top-left quadrant of the display screen. The monitor 130 renders “Image 2” associated with image signal S2 applied to input port P2 in the bottom-left quadrant of the display screen. The monitor 130 renders “Image 3” associated with image signal S3 applied to input port P3 in the top-right quadrant of the display screen. The monitor 130 renders “Image 4” associated with image signal S4 applied to input port P4 in the bottom-right quadrant of the display screen.

In this example, each of the image signals S1-S4 may be configured to have a certain pixel resolution and frame rate. For example, each of the signals S1-S4 may be compliant with the Digital Visual Interface (DVI) specification in accordance with a single-link transmission. As a more specific example, the DVI single-link image resolution may be 1920×1080 (i.e., 1920 pixels per horizontal line and 1080 horizontal lines). Accordingly, all of the images 1-4 on the monitor screen have the 1920×1080 resolution. The total resolution for the entire monitor screen is then 3840×2160 (e.g., 4×(1920×1080)). It shall be understood that each of the image signals may be configured in accordance with different resolutions and formats, such as High-Definition Multimedia Interface (HDMI), DisplayPort, as well as others.

Recently, monitors configured to display medical images have been designed to accept signals in formats different than those capable of being produced by the image manager 120. For example, such monitors may include only two input ports for accepting image signals that are rendered in the left-half and right-half of the monitor screen, respectively. As a more specific example, such monitors may include two dual-link DVI inputs that accepts image signals each having a pixel resolution of 1920×2160 (i.e., 1920 pixels per horizontal line and 2160 horizontal lines).

Accordingly, medical personnel that desire using the new monitors would require replacing the image manager 120 with one that is capable of producing output signals in accordance with the format accepted by the new monitors. However, such image managers are expensive. To minimize costs while using the new monitors, it would be desirable to maintain the image manager 120 in the system, and provide an interface to convert the output signals S1-S4 of the image manager to a format accepted by the new monitor.

Furthermore, it would be desirable for such interface to be easily connectable to such new monitor in a manner that the interface seems as an integral part of the monitor. Additionally, for monitor authentication, the interface would send authentication information pertaining to the old or legacy monitor to the image manager 120 so that the image manager operates as if it is connected to the old monitor. Such systems that are capable of implementing the aforementioned functionality are described below with reference to various exemplary embodiments.

FIG. 2 illustrates a block diagram of another exemplary medical imaging system 200 in accordance with another aspect of the disclosure. Similar to system 100, the system 200 includes the set of medical imaging devices 110-1 to 110-N coupled to the image manager 120, which, in turn, is configured to generate the output signals S1-S4.

In contrast to system 100, the system 200 includes a monitor 230 that includes only two image signal input ports MP1 and MP2. For example, the input ports MP1 and MP2 may be configured to receive image signals to render images 1-2 on the left-half and right-half of the monitor screen, respectively. As a more specific example, the input ports MP1 and MP2 may be configured as DVI dual-link input ports, which may be configured to receive signals each with pixel resolution of 1920×2160.

As discussed above with reference to system 100, the image manager 120 may be configured to produce image signals S1-S4 not all accepted by the two input ports MP1 and MP2 of the monitor 230 in a manner to completely render an image on the entire monitor screen. As a specific example, the output image signals S1-S4 may each be DVI single-link compliant with a resolution of 1920×1080.

To address this potential incompatibility, the system 200 further includes a monitor interface 240. The interface 240 includes four (4) input ports IIP1-IIP4 configured to receive the image signals S1-S4 from the image manager 120, respectively. As a specific example, the input ports IIP1-IIP4 may be configured as DVI single-link ports configured to accept the 1920×1080 resolution input signals S1-S4, respectively. The signals S1-S4 may be transmitted from the image manager 120 to the interface 240 by way of appropriate cabling, such as DVI cables or others.

The interface 240 further includes two (2) output ports IOP1-IOP2 configured to couple (e.g., compliantly mate) with the input ports MP1-MP2 of the monitor 230, respectively. The interface 240 is configured to multiplex two of the received image signals (e.g., S1 and S2) to generate an output image signal S12 to be applied to the input port MP1 of the monitor 230 via the output port IOP1. Similarly, the interface 240 is configured to multiplex another two of the received image signals (e.g., S3 and S4) to generate an output image signal S34 to be applied to the input port MP2 of the monitor 230 via the output port IOP2.

As a specific example, the interface 240 is configured to multiplex the two DVI single-link signals S1-S2 (e.g., each having a resolution of 1920×1080) to generate the output signal S12 as a DVI dual link signal having a resolution of 1920×2160. As the image signal S12 is applied to the input port MP1 of the monitor 230, the monitor 230 renders image 1 on the left-half of the monitor screen based on the image signal S12. Similarly, the interface 240 is configured to multiplex the other two DVI single-link signals S3-S4 (e.g., each having a resolution of 1920×1080) to generate the output signal S34 as a DVI dual link signal having a resolution of 1920×2160. As the image signal S34 is applied to the input port MP2 of the monitor 230, the monitor 230 renders image 2 on the right-half of the monitor screen based on the image signal S34.

Thus, the system 200 allows the use of a new-type monitor that includes, for example, two DVI dual link input ports MP1 and MP2, while not requiring the use of a new image manager. As discussed above, this would be a substantial cost savings for medical institutions that convert or upgrade from the system 100 to the system 200. Additionally, as discussed further below, the interface 240 may also provide authentication information to the image manager 120 in a manner that the image manager operates as if it is connected to the old monitor 130.

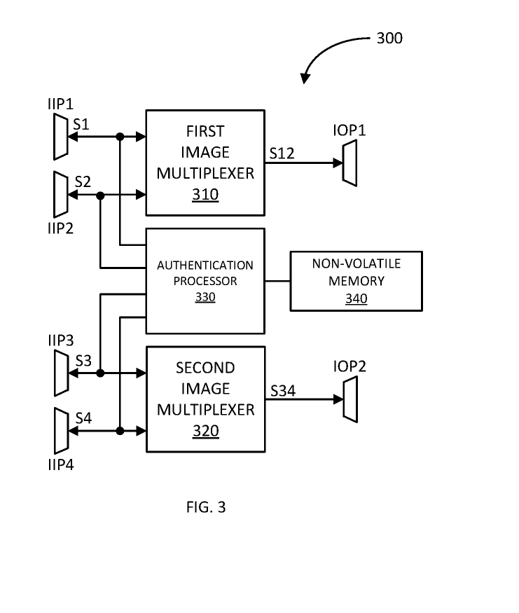

FIG. 3 illustrates a block diagram of an exemplary monitor interface 300 in accordance with another aspect of the disclosure. The interface 300 may be an exemplary detailed implementation of the interface 240 previously discussed. Similar to interface 240, the interface 300 includes input ports IIP1-IIP4 configured to receive the image signals S1-S4 from the image manager 120, respectively. Also, similar to interface 240, the interface 300 includes output ports IOP1-IOP2 configured to output image signals S12 and S34, respectively. As previously discussed, the output ports IOP1-IOP2 are configured to interface (e.g., compliantly mate) with the input ports MP1-MP2 of the monitor 230, respectively.

The interface 300 includes a first image multiplexer 310, a second image multiplexer 320, an authentication processor 330, and a non-volatile memory 340. The first image multiplexer 310 is configured to receive the image signals S1 and S2 from the respective input ports IIP1-IIP2, and multiplex the signals to generate the output image signal S12. The first image multiplexer 310 sends the output image signal S12 to the output port IOP1. Similarly, the second image multiplexer 320 is configured to receive the image signals S3 and S4 from the respective input ports IIP3-IIP4, and multiplex the signals to generate the output image signal S34. The second image multiplexer 320 sends the output image signal S34 to the output port IOP2.

The authentication processor 330 is configured to send authentication information to the image manager 120 by way of the input ports IIP1-IIP4. The non-volatile memory 340 is configured to store the authentication information. The authentication information may pertain to a monitor (e.g., monitor 130) different than the monitor to which the interface 300 is connected. This allows the image manager 120 to operate as if it is connected to the “old” monitor (e.g., monitor 130). The authentication information may include one or more of the following: monitor name (e.g., manufacturer), model number, other identification, or image capability (e.g., capable of accepting four (4) simultaneous DVI single-link signals). Thus, in response to a request for the authentication information sent by the image manager 120, the authentication processor 330 accesses the information from the memory 340 and sends it to the image manager via the input ports IIP1-IIP4.

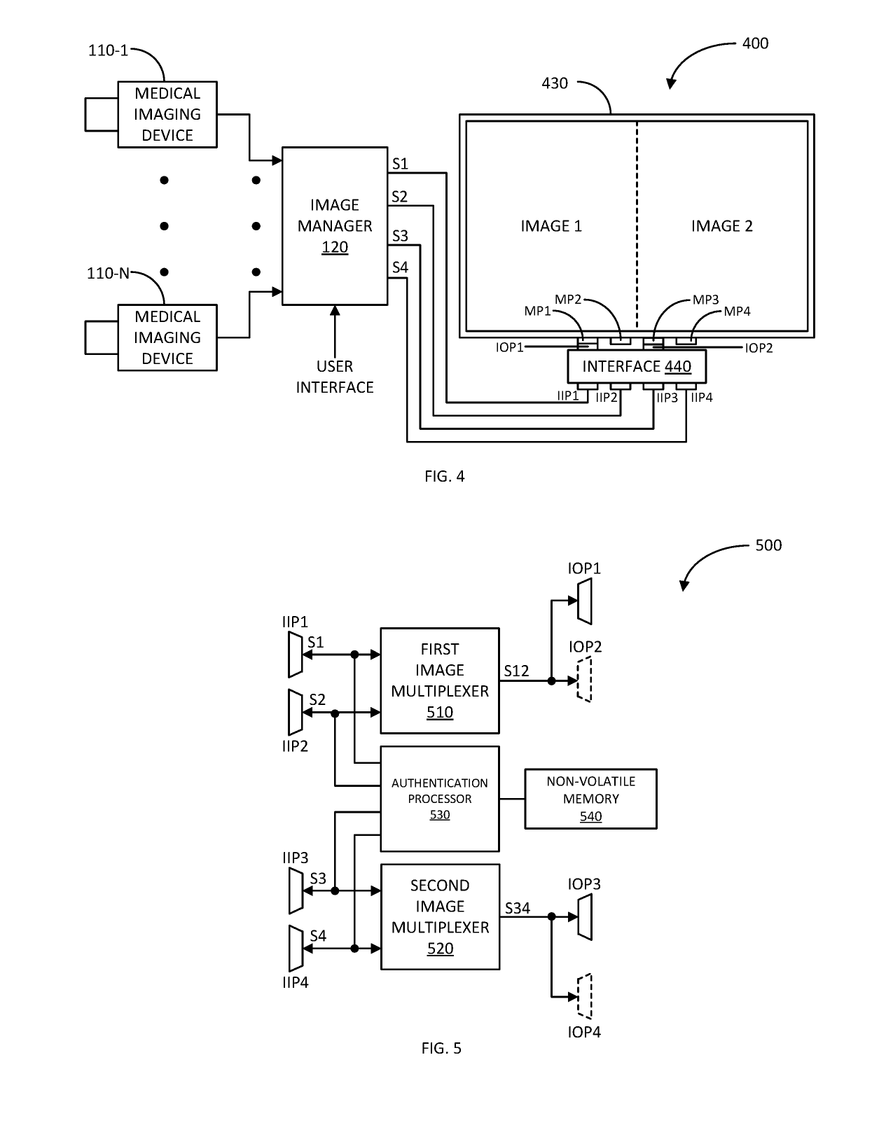

FIG. 4 illustrates a block diagram of another exemplary medical imaging system 400 in accordance with another aspect of the disclosure. The system 400 is similar to system 200 (and includes many of the same elements as indicated by the same reference numbers), but uses a different monitor and a different interface. In particular, the system 400 includes a monitor 430 that includes two selectable pairs of input ports MP1/MP3 and MP2/MP4. The monitor 430 is configured to render images 1-2 on the left-half and right-half of the monitor screen based on a pair of signals received via the selected pair of input ports MP1/MP3 or MP2/MP4. The monitor 430 may include a user interface to allow a user to select one of the input port pairs for image rendering purposes. The monitor 430 may default to selecting one of the pairs (without specific user selection), such as input port pair MP1/MP3.

Additionally, the system 400 includes an interface 440 that includes two output ports IOP1 and IOP2 configured to interface (e.g., compliantly mate) with one of the pairs of input ports (e.g., the default MP1/MP3) of the monitor 430. Similar to the interface 240, the interface 440 includes input ports IIP1-IIP4 configured to receive image signals S1-S4 from the image manager 120, respectively. Also, similar to the interface 240, the interface 440 multiplexes input image signals S1-S2 to generate output image signal S12 for sending to the monitor 430 via ports IOP1 and MP1. Similarly, the interface 440 multiplexes input image signals S3-S4 to generate output image signal S34 for sending to the monitor 430 via ports IOP3 and MP3.

FIG. 5 illustrates a block diagram of another exemplary image manager to monitor interface 500 in accordance with an aspect of the disclosure. The interface 500 may be an exemplary detailed implementation of the interface 440. The interface 500 includes a first image multiplexer 510 configured to multiplex signals S1-S2 received via the input ports IIP1-IIP2 to generate output image signal S12. The output image signal S12 may be applied to output port IOP1 for transmission to the attached monitor 430. The output image signal S12 may also be sent to a reserved port IOP2, which may not include a physical port (e.g., DVI connector) for connection to the monitor. However, the reserved port IOP2 may be configured to optionally receive a physical port to allow the output signal S12 to be transmitted to a device by way of the physical port if it exists.

Similarly, the interface 500 includes a second image multiplexer 520 configured to multiplex signals S3-S4 received via the input ports IIP3-IIP4 to generate output image signal S34. The output image signal S34 may be applied to output port IOP3 for transmission to the attached monitor 430. The output image signal S34 may also be sent to a reserved port IOP4, which may not include a physical port for connection to the monitor. However, the reserved port IOP4 may be configured to optionally receive a physical port to allow the output signal S34 to be transmitted to a device by way of the physical port if it exists.

Similar to interface 300, the interface 500 includes an authentication processor 530 and an associated non-volatile memory 540 for transmitting authentication information to the image manager 120 by way of the input ports IIP1-IIP2, as previously discussed.

FIG. 6 illustrates a rear perspective view of an exemplary monitor 630 and a monitor interface 640 in accordance with another aspect of the disclosure. The monitor 630 includes a central rear surface 632, a surface 635 upon which the input ports of the monitor 630 (not shown) are supported. For example, in the case of monitor 230, the input ports MP1 and MP2 are supported upon surface 635. In the case of monitor 430, the input ports MP1-MP4 are supported upon surface 635. Additionally, the monitor 630 includes a perimeter rear surface 636 surrounding the central rear surface 632.

The interface 640 includes a housing 642 for enclosing the various components as previously described, such as the first and second image multiplexers, authentication processor, and non-volatile memory. As viewed from the side, the housing 642 may be L-shaped with a flange 646 attached to the top of a portion 644 of the L-shaped housing. The flange 646 is configured to lie substantially flat upon a lower and central portion of the central rear surface 632 of the monitor 630. The flange 646 includes a set of thru-holes 634 that register with a set of threaded holes (not shown) on the central rear surface 632 of the monitor 630. A set of threaded screws (not shown) are configured to be threaded into the set of threaded holes via the set of thru-holes 634 to secure the flange 646 onto the central rear surface 632, respectively.

The interface 640 includes a minor flange 650 with a thru hole that registers with a threaded hole (not shown) on a lower and central portion the perimeter rear surface 636. A threaded screw (not shown) is configured to be threaded into the threaded hole via the thru-hole of the minor flange 650 to secure the minor flange onto the perimeter rear surface 636. In this configuration, the interface 640 is secured to the monitor 630 in a manner that an upper surface (hidden in FIG. 6) of the portion 644 of the L-shaped housing 640 is substantially flushed (e.g., substantially parallel and adjacent to each other) with the connector surface 635 of the monitor 630.

As shown, the interface 640 includes input physical ports (e.g., DVI connectors graphically represented) supported on a lower surface of the portion 644 of the L-shaped housing 640. The input physical ports may be evenly and centrally distributed upon the lower surface of the portion 644 of the L-shaped housing 640. The interface 640 may include port identification indicia (not shown) disposed on a rear surface 642 of a portion 645 of the L-shaped housing. The port identification indicia may includes four (4) identifiers juxtaposed proximate the corresponding input physical ports. The interface 640 includes output physical ports (not shown as they are hidden in FIG. 6) disposed on the upper surface of the portion 644 of the L-shaped housing. The output physical ports compliantly mate with corresponding input ports (not shown as they are hidden in FIG. 6) disposed on the connector surface 635 of the monitor 630.

The interface 640 includes a power cord for connection to an input power port of the monitor 630. A separate power cord as shown may be connected to the interface 640. In this way, power is supplied to the monitor 630 by way of the interface 640. Thus, as illustrated, the interface 640 appears as an integral part of the monitor 630.

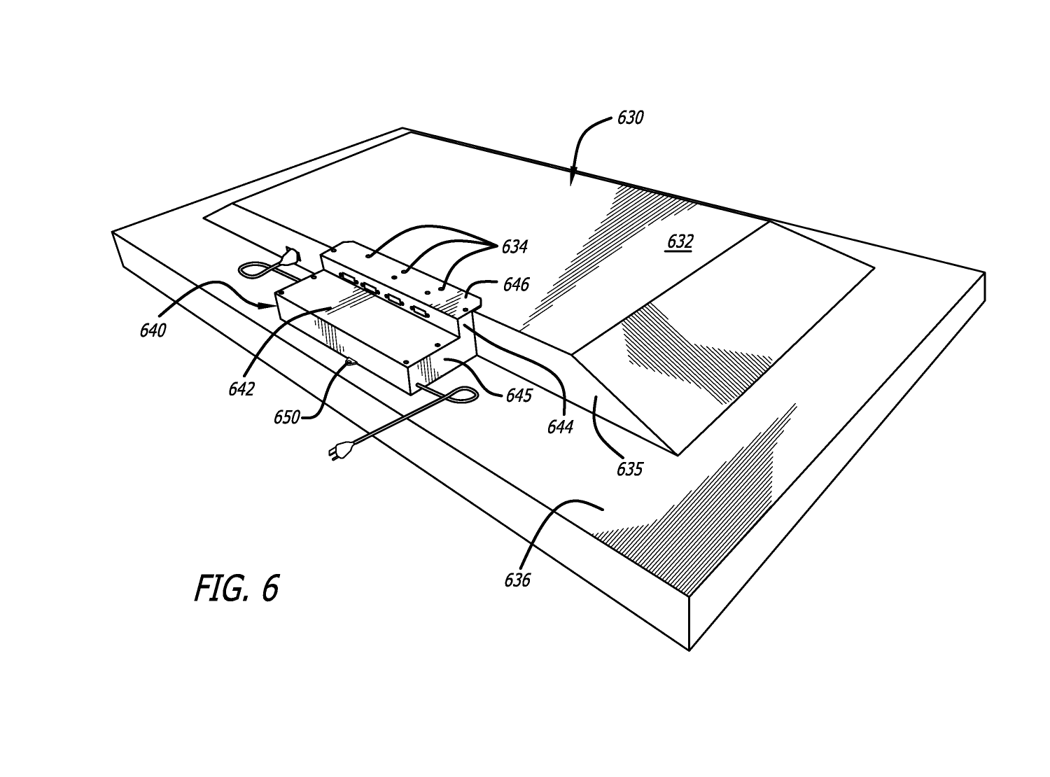

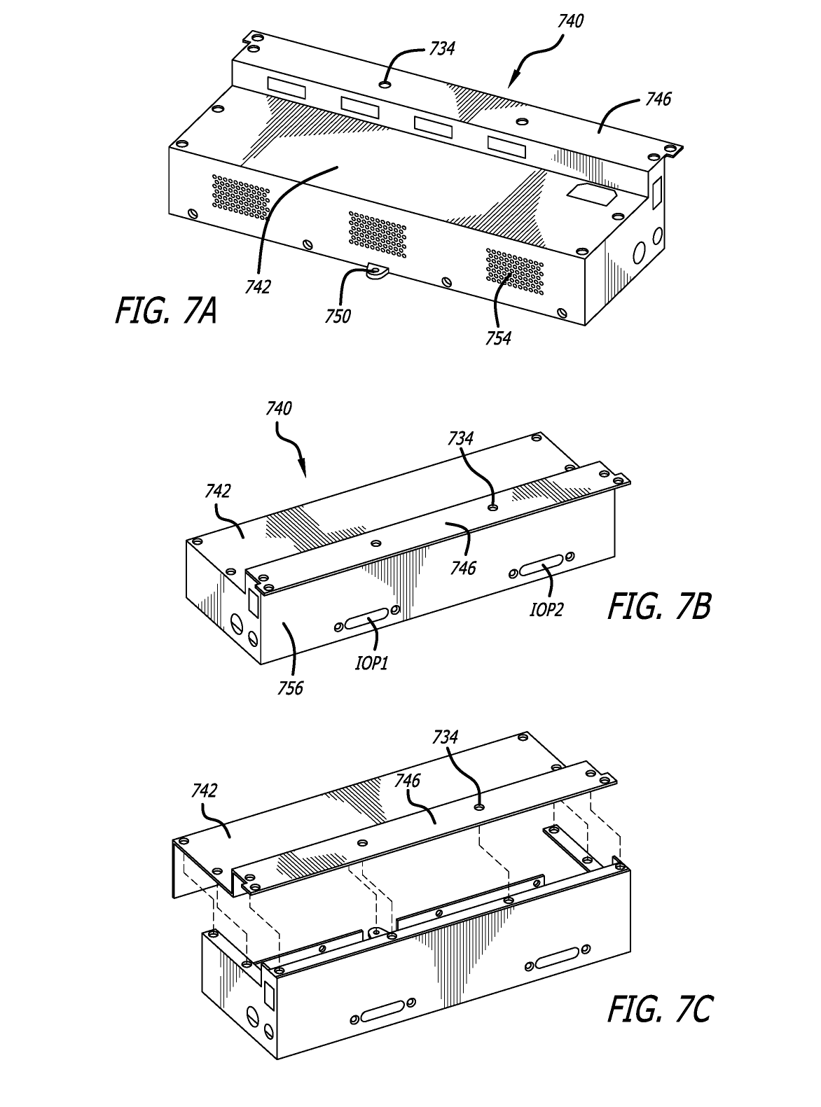

FIGS. 7A-7C illustrate front perspective, rear perspective, and rear disassembled views of an exemplary monitor interface 740 in accordance with another aspect of the disclosure. The interface 740 may be a detailed physical implementation of the interface 240 previously discussed. The interface 740 is similar to interface 640 (and includes many of the same elements as indicated by the same reference numbers with the most significant digital being a “7” instead of a “6.”). In this exemplary embodiment, the housing 740 includes vents 754 to allow heat to escape from within the housing to maintain the components at desirable operating temperatures. Also, as shown, the interface 740 includes output physical ports IOP1 and IOP2 disposed on a front surface 756 of a vertical portion of an L-shaped housing 742.

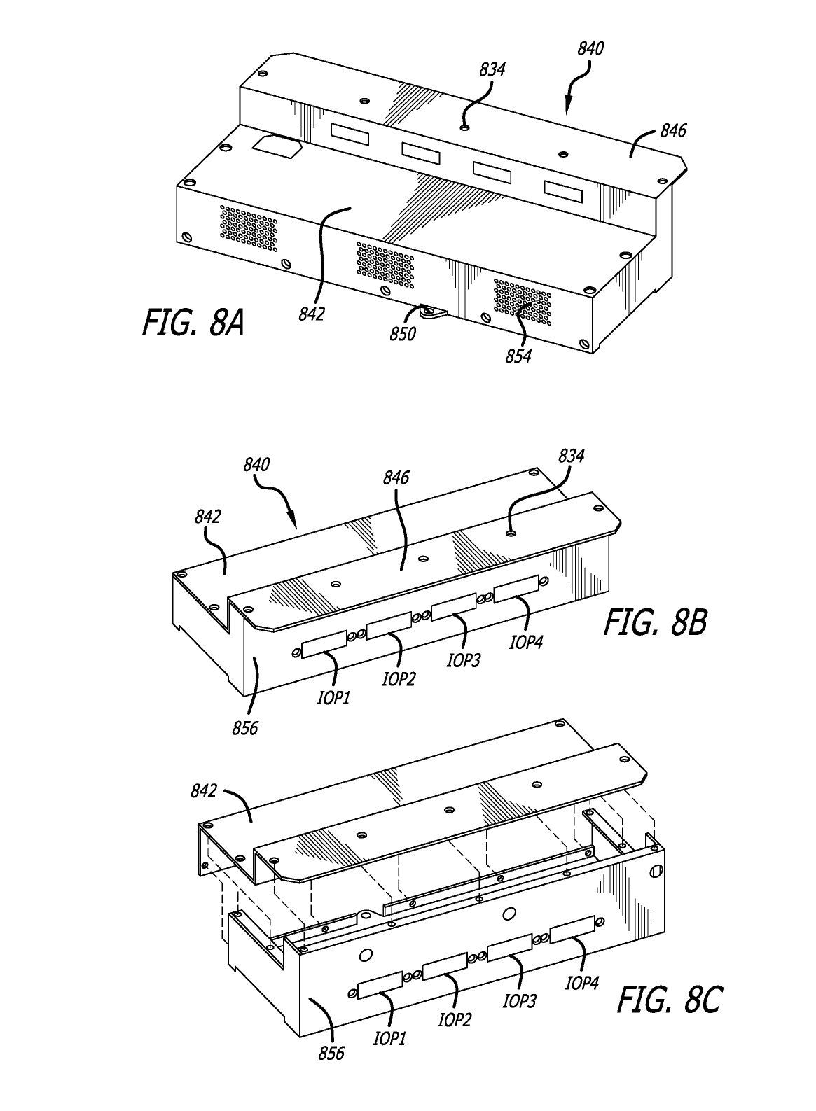

FIGS. 8A-8C illustrate rear perspective, front perspective, and front disassembled views of an exemplary image manager to monitor interface 840 in accordance with another aspect of the disclosure. The interface 840 may be a detailed physical implementation of the interface 440 previously discussed. The interface 840 is similar to interface 640 (and includes many of the same elements as indicated by the same reference numbers with the most significant digital being a “8” instead of a “6.”). In this exemplary embodiment, the housing 840 also includes vents 854 to allow heat to escape from within the housing to maintain the components at desirable operating temperatures. Additionally, as shown, the interface 840 includes output ports IOP1-IOP4 (two of which may be configured with physical connectors) disposed on a front surface 856 of a vertical portion of an L-shaped housing 842.

The previous description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. Thus, the disclosure is not intended to be limited to the examples described herein but is to be accorded the widest scope consistent with the principles and novel features disclosed herein.

What is claimed is:

1. An interface, comprising:
a housing configured to be securely attached to a first monitor;
a set of input ports configured to receive a set of input image signals from an image manager, wherein the set of input ports are disposed on a first surface of the housing;
at least one image multiplexer configured to multiplex at least a pair of the set of input image signals to generate at least one output image signal, wherein the at least one image multiplexer is situated within the housing; and
at least one output port disposed on a second surface of the housing, wherein the at least one output port is configured to compliantly mate with at least one input port of the first monitor while the housing is securely attached to the first monitor, wherein the at least one output port is configured to produce the at least one output image signal, respectively.

2. The interface of claim 1, further comprising an authentication processor configured to transmit authentication information pertaining to a second monitor to the image manager.

3. The interface of claim 2, wherein the authentication processor is configured to transmit the authentication information to the image manager via at least one of the set of input ports.

4. The interface of claim 2, wherein the authentication processor is situated within the housing.

5. The interface of claim 2, further comprising a non-volatile memory configured to store the authentication information.

6. The interface of claim 5, wherein the non-volatile memory is situated within the housing.

7. The interface of claim 1, wherein the set of input image signals are configured to cause the second monitor to simultaneously render corresponding images on separate portions of a screen of the second monitor, respectively.

8. The interface of claim 7, wherein the at least one output image signal is configured to cause the first monitor to simultaneously render at least one corresponding image on at least one portion of a screen of the first monitor, respectively.

9. The interface of claim 8, wherein a quantity of the at least one portion of the screen of the first monitor is less than a quantity of the separate portions of the screen of the second monitor.

10. The interface of claim 1, wherein each of the set of input image signals are configured to have a first pixel resolution, and wherein each of the at least one output image signal is configured to have a second pixel resolution different than the first pixel resolution.

11. The interface of claim 10, wherein the first pixel resolution is substantially half of the second pixel resolution.

12. The interface of claim 1, wherein each of the set of input ports comprises a single-link Digital Visual Interface (DVI) port, and wherein each of the at least one output port comprises a dual-link DVI port.

13. The interface of claim 1, wherein the at least one output image signal comprises first and second output image signals, and wherein the at least one image multiplexer comprises first image multiplexer configured to multiplex first pair of the set of input images signals to generate the first output image signal and second image multiplexer configured to multiplex second pair of the set of input images signals to generate the second output image signal.

14. The interface of claim 13, wherein the at least one output port comprises first and second pair of output ports, and further comprising:
a user interface configured to generate first and second control signals based on a user input;
a first switch configured to route the first output image signal to one of the first pair of the output ports or to one of the second pair of the output ports based on the first control signal; and
a second switch configured to route the second output image signal to the other of the first pair of the output ports or to the other of the second pair of the output ports based on the second control signal.

15. The interface of claim 1, wherein the housing is securely attached to the first monitor in a manner that the housing substantially appears to be integral with the first monitor.

16. A medical imaging system, comprising:
a set of medical imaging devices configured to generate first and second pairs of input image signals concerning a subject undergoing a medical treatment or diagnosis;
an image manager configured to route the first and second pairs of input image signals from a first set of inputs ports to a first set of output ports based on a user input;
an interface, comprising:
a housing;
a second set of input ports coupled to the first set of output ports of the image manager, respectively, wherein the second set of input ports are disposed on a first surface of the housing;
a pair of image multiplexers disposed within the housing, wherein the pair of image multiplexers is configured to multiplex the first and second pairs of input image signals to generate first and second output image signals, respectively; and
first and second output ports disposed on a second surface of the housing, wherein the first and second output ports are configured to produce the first and second output image signals, respectively; and
a first monitor including first and second input ports compliantly mated with the first and second output ports of the interface, respectively, wherein the first monitor is configured to simultaneously render first and second images on first and second portions of a screen based on the first and second output image signals, respectively.

17. The medical imaging system of claim 16, wherein the interface further comprises an authentication processor configured to transmit authentication information pertaining to a second monitor to the image manager.

18. The medical imaging system of claim 17, wherein of the input image signals of the first and second pairs are configured to cause the second monitor to simultaneously render corresponding images on separate portions of a screen of the second monitor, respectively.

19. The medical imaging system of claim 18, wherein the first and second output image signals are configured to cause the first monitor to simultaneously render corresponding images on separate portions of a screen of the first monitor, respectively.

20. A medical imaging apparatus, comprising:
an interface, comprising:
a housing;
a first set of input ports configured to receive a set of input image signals, respectively, wherein the first set of input ports are disposed on a first surface of the housing;
a set of image multiplexers configured to multiplex pairs of the set of input image signals to generate a set of output image signals, respectively, wherein the set of image multiplexers are enclosed within the housing; and
a set of output ports configured to produce the set of output image signals, respectively, wherein the set of output ports are disposed on a second surface of the housing;
a monitor including a second set of input ports compliantly mated with the set of output ports of the interface, respectively, wherein the monitor is configured to simultaneously render a set of images on separate portions of a screen based on the set of output image signals, respectively.
